(19) United States
(12) Patent Application Publication
(10) Pub. No.: US 2011/0124706 A1
(43) Pub. Date: May 26, 2011

(54) SOCS3 Inhibition Promotes CNS Neuron Regeneration

(76) Inventors: Zhigang He, Boston, MA (US); Patrice D. Smith, Ottawa (CA); Fang Sun, Boston, MA (US)

(21) Appl. No.: 12/626,351

(22) Filed: Nov. 25, 2009

(51) Int. Cl. A61K 31/7105; A61P 25/00

(52) U.S. Cl. 514/44 A; 514/44 R

(57) ABSTRACT

Regeneration of injured CNS neurons in a mammal in need thereof is promoted by delivering directly to the body of the injured CNS neurons, such as with an intracerebral or intraretinal cannula, an effective amount of a specific inhibitor of SOCS3.

This invention was made with government support under Grant No. R01NS051788 awarded by the NIH. The government has certain rights in this invention.

### BACKGROUND OF THE INVENTION

The field of the invention is promoting CNS neuron regeneration by administering at the cell body a specific inhibitor of SOCS3.

Axon regeneration failure accounts for permanent functional deficits following CNS injury in adult mammals. However, the underlying mechanisms that control axon regeneration in the adult CNS remain elusive. In our systematic efforts to assess axon regeneration following the model optic nerve injury in different mutant mouse lines, we discovered that deletion of suppressor of cytokine signaling 3 (SOCS3), in adult retinal ganglion cells (RGCs), promotes robust regeneration of injured optic nerve axons. This axon regeneration promoting effect is efficiently blocked in SOCS3-gp130 double knockout mice, indicating that SOCS3 deletion promotes axon regeneration via the gp130-dependent signaling pathway. Consistently, a transient up-regulation of ciliary neurotrophic factor (CNTF) was observed within the retina following optic nerve injury. In addition, intravitreal application of CNTF further enhances the extent of axon regeneration observed in SOCS3 knockout mice. Our results indicate that compromised responsiveness to injury-induced growth factors in mature neurons contribute significantly to regeneration failure. Thus, we developed practical methods to modulate negative signaling regulators in order to render neurons responsive to growth-promoting factors as an efficient strategy to promote axon regeneration after CNS injury. Here we disclose that specific inhibition of SOCS3 promotes regeneration of injured CNS neurons when delivered directly to the cell bodies, as opposed to distal sites of injury or lesion, and the regeneration is enhanced with coadministered CNTF.

Park et al. (Mol Cell Neurosci 41, 313-324, 2009) report cytokine-induced SOCS expression is inhibited by cAMP analogue: impact on regeneration in injured retina.

Girolami et al. (Exp Neurol. 2009 Jul. 2. Epub ahead of print) report differential expression and potential role of SOCS1 and SOCS3 in Wallerian degeneration in injured peripheral nerve.

Okada et al. (Nature Medicine 12(7) July 2006) report that conditional ablation of Stat3 or Socs3 discloses a dual role for reactive astrocytes after spinal cord injury.

Lee et al. (Restor Neurol Neurosci. 2008; 26(2-3): 175-182) review axon regeneration after spinal cord injury: insight from genetically modified mouse models.

### SUMMARY OF THE INVENTION

The invention provides methods and compositions for treating a mammal in need thereof having a CNS injury. In one aspect, the general method comprises the step of: delivering directly to the body of an injured CNS neuron of the mammal an effective amount of a specific inhibitor of suppressor of cytokine signaling 3 (SOCS3), and optionally further comprising detecting a resultant improved recovery from the injury.

In another aspect, the invention provides methods comprising the step of: delivering directly to the body of the injured CNS neuron an effective amount of a specific inhibitor of suppressor of cytokine signaling 3 (SOCS3), wherein the delivered SOCS3 inhibitor promotes regeneration of the injured CNS neuron, and optionally detecting a resultant promoted regeneration of the injured CNS neuron.

The invention encompass all combinations of particular embodiments, including wherein:

the delivery is intracortical, intracerebral, or intra-retinal;

the delivery is made to cerebrospinal fluid at the body of the injured CNS neuron;

the delivery is effected with a cannula;

the inhibitor is SOCS3-specific hpRNA or siRNA;

the inhibitor is antisense SOCS3 or dominant negative SOCS3;

the neuron is injured as a result of trauma or stroke; and/or

the method further comprises the step of delivering to the body of the injured neuron cytokine CNTF sufficient to activate gp 130 in the neuron.

In another aspect the invention provides compositions specifically tailored to implementing the subject methods, such as an intracerebral or intraretinal cannula loaded with an amount of a specific inhibitor of SOCS3 effective to promote regeneration of an injured CNS neuron when delivered directly to the body of the injured CNS neuron in a mammal in need thereof.

### DETAILED DESCRIPTION OF SPECIFIC EMBODIMENTS OF THE INVENTION

In one aspect, the invention provides methods for treating a mammal in need thereof having a CNS injury comprising the step of: delivering directly to (or contacting) the body of an injured CNS neuron of the mammal an effective amount of a specific inhibitor of suppressor of cytokine signaling 3 (SOCS3), wherein a resultant improved recovery from the injury is obtained. In another aspect the invention provides a method for promoting regeneration of an injured CNS neuron in a mammal in need thereof, comprising the step of: delivering directly to (or contacting) the body of the injured CNS neurons an effective amount of a specific inhibitor of SOCS3, wherein the delivered SOCS3 inhibitor promotes regeneration of the injured CNS neuron.

The subject methods and compositions are applicable to SOCS3-expressing injured CNS neurons, particularly brain and optic nerve neurons under SOCS3-mediated regeneration inhibition, and which may be injured as a result, for example, of traumatic injury, optic nerve injury or disorder, brain injury, stroke, chronic neurodegeneration such as caused by neurotoxicity or a neurological disease or disorder (e.g. Huntington's disease, Parkinson's disease, Alzheimer's disease, multiple system atrophy (MSA), etc.). In a particular embodiment, the lesion results from acute or traumatic injury such as caused by contusion, laceration, acute spinal cord injury, etc. In specific embodiments, the injured neuron is in CNS white matter, particularly white matter that has been subjected to traumatic injury.

The timing and duration of the deliver or contact is tailored to the indication, injury and inhibitor. In certain embodiments, the delivery step is initiated within 30, 14 or 7 days, preferably 96, 72, 48, 24, or 12 hours of the injury. In various embodiments, the delivery step is initiated, and/or treatment is continued, more than 5, 7, 14, 30, or 60 days.

Preferred target mammals include human, companion animal (e.g. dog, cat), livestock (e.g. bovine, equine, porcine, goat), rodent (rat or mouse), primate and mammalian models for CNS injury.

The inhibitor specifically binds or competes with the SOCS-3 gene, transcript or translate (protein). Suitable inhibitors include SOCS3-specific polynucleotides and PNAs targeting the SOCS3 gene or transcripts, and include SOCS3-specific hpRNA, siRNA, and antisense polynucleotides. Materials and methods for making and using such polynucleotides are known in the art or otherwise exemplified below, including design and cloning strategies for constructing suitable SOCS3 shRNA expression vectors (e.g. McIntyre et al., BMC Biotechnol. 2006; 6:1), and suitable antisense SOCS3 cDNAs (Owaki, et al., J. Immunol. 2006 Mar. 1; 176(5):2773-80). Suitable SOCS3-specific polynucleotides targeting the SOCS3 gene or transcripts are also commercially available from several vendors including OriGene (Rockville Md.) such as vector pRFP-C-RS and pGFP-V-RS, human 29mer shRNA constructs against SOCS3 in pRFP-C-RS and pGFP-V-RS vectors, respectively. SOCS3 specific siRNA is also widely commercially available, e.g. Santa Cruz Biotechnology, Inc.

Suitable inhibitors also include SOCS3-based polypeptides like dominant negative SOCS3 peptides and proteins, such as SOCS3 (F25A) (e.g. Owaki, et al., J. Immunol. 2006 Mar. 1; 176(5):2773-80), which contains a point mutation in the kinase inhibitory region of SOCS3.

SOCS3-specific antibody and intrabody inhibitors may also be used, such as have been intrabodies for the therapeutic suppression of a variety of neurodegenerative pathologies, e.g. Messer et al. Expert Opin Biol Ther. 2009 September; 9(9):1189-97.

The structural determination of SOSC3 has also facilitated development of small-molecule SOCS3 specific inhibitors, e.g. Babon et al., J Mol Biol. 2009 Mar. 20; 387(1):162-74; Babon et al., Mol Cell 2006 Apr. 21; 22 (2) 205-16. Structure-based SAR yield chemically diverse small molecule SOCS3 inhibitors at micro- and nanomolar activity.

SOCS3 inhibition is readily assayed by specific techniques, such as immunocytochemistry. Because SOCS3 up-regulation occurs after CNTF treatment inhibitors of SOCS3 (expression or activity) allow sustained p-STAT3 levels, and SOCS3 inhibition may be measured by STAT3 activation. For example, COS cells can be treated with CNTF and monitored for sustained phosph-STAT3 signals. In another embodiment, cultured CNS neurons can be incubated in serum-free medium with or without serially-diluted inhibitor, e.g. for 6 hr. The cells are then incubated with a polyclonal antibody against phospho-STAT3, such as Tyr705 (Cell Signaling Technology, Danvers, Mass.); see, e.g. Liu et al., J Neurosci, September 2001, 21(17) RC164, 1-5.

Those skilled in the art can also employ established methods for increasing the efficiency and/or efficacy of the delivery, such as use of a cell-penetrating peptide for enhanced delivery of nucleic acids and drugs to retinal neurons, e.g. Johnson et al., Mol Ther. 2008 January; 16(1):107-14.

The inhibitor can be administered to the injured neuron in combination with, or prior or subsequent to, other treatments such as the use of anti-inflammatory or growth or trophic factors, etc. In various other embodiments, the activator is administered in combination with trophic and/or growth factors such as NT-3 (Piantino et al, Exp Neurol. 2006 October; 201(2):359-67), inosine (Chen et al, Proc Natl Acad Sci USA. (2002) 99:9031-6; US Pat Publ 20050277614), oncomodulin (Yin et al, Nat Neurosci. (2006) 9:843-52; US Pat Pubs 20050054558 & 20050059594; and U.S. Pat. No. 6,855,690), etc. In a particular embodiment the method further comprises the step of delivering to the body of the injured neuron cytokine CNTF sufficient to activate gp 130 in the neuron.

The inhibitor is contacted with the neuron body using a suitable delivery method and treatment protocol sufficient to promote regeneration of the subject neuron, and tailored to the particular target neuron, inhibitor and indication. Exemplary deliveries include intracortical, intracerebral, and intra-retinal, as well as deliveries made to cerebrospinal fluid at the body of the injured CNS neuron. Exemplary delivery methods include intracerebral microinfusion (ICM), intracerbebral cannulae (e.g. Kenny et al. Neuropsychopharmacol. 2009 January; 34(2): 266-281), and retinal delivery (e.g. Maguire et al., N Engl J. Med. 2008 May 22; 358(21):2240-8).

In a specific embodiment, the inhibitor is contacted with the neuron body using an implantable device that contains the inhibitor, such as an implantable device or cannula, preferably specifically adapted for delivery to a CNS neuron body. Examples of devices include solid or semi-solid devices such as controlled release biodegradable matrices, fibers, pumps, stents, absorbable gelatin (e.g. Gelfoam), etc. The device may be loaded with premeasured, discrete and contained amounts of the inhibitor sufficient to promote regeneration. In a particular embodiment, the device provides continuous contact of the neuron with the inhibitor at nanomolar or micromolar concentrations, preferably for at least 2, 5, or 10 days.

In particular embodiments the method further comprises a detecting step, such as the step of detecting a resultant improved recovery from the injury, or detecting a resultant promoted regeneration of the injured CNS neuron, which can be detected directly using imaging methodologies such as MRI, or indirectly or inferentially, such as by neurological examination showing improvement in the targeted neural function. The detecting step may occur at any time point after initiation of the treatment, e.g. at least one day, one week, one month, three months, six months, etc. after initiation of treatment. In certain embodiments, the detecting step will comprise an initial neurological examination and a subsequent neurological examination conducted at least one day, week, or month after the initial exam. Improved neurological function at the subsequent exam compared to the initial exam indicates resultant axonal regeneration. The specific detection and/or examination methods used will usually be based on the prevailing standard of medical care for the particular type of neuron injury being evaluated (i.e. trauma, neurodegeneration, etc.).

In another aspect the invention provides surgical device loaded with an amount of a specific inhibitor of SOCS3 effective to promote regeneration of an injured CNS neuron when delivered directly to the body of the injured CNS neuron in a mammal in need thereof. Exemplary such devices include intracerebral or intraretinal cannulae, and inhibitor-eluting or inhibitor-impregnated CNS implantable solid or semi-solid devices (supra). Examples of such CNS implantable devices include polymeric microspheres (e.g. see Benny et al., Clin Cancer Res. (2005) 11:768-76) or wafers (e.g. see Tan et al., J Pharm Sci. (2003) 4:773-89), biosynthetic implants used in tissue regeneration after spinal cord injury (reviewed by Novikova et al., Curr Opin Neurol. (2003) 6:711-5), biodegradable matrices (see e.g. Dumens et al., Neuroscience (2004) 125:591-604), biodegradable fibers (see e.g. U.S. Pat. No. 6,596,296), osmotic pumps, stents, absorbable gelatins (see e.g. Doudet et al., Exp Neurol. (2004) 189:361-8), etc. Preferred devices are particularly tailored, adapted, designed or designated for CNS implantation. The device may contain one or more additional agents used to promote or facilitate neural regeneration, such as a nerve growth factor, trophic factor, or hormone that promotes neural cell survival, growth, and/or differentiation, such as brain-derived neurotrophic factor (BDNF), ciliary neurotrophic factor (CNTF), nerve growth factor (NGF), inosine, oncomodulin, NT-3, etc.

### Example 1

### SOCS3 Deletion Promotes Optic Nerve Regeneration in Adult Mice

We applied optic nerve crush injury to assess axon growth ability in adult CNS in different conditional floxed mice (Park et al., 2008 Science 322, 963-966). Intravitreal application of recombinant adeno-associated viruses expressing Cre (AAVCre) results in Cre-dependent reporter expression in more than 90% of RGCs. Regeneration was assessed in the injured optic nerve axons by using cholera toxin beta subunit (CTB), an anterograde tracer, which was injected into the vitreous of the retina following optic nerve injury two days prior to histological analyses. Neuronal survival was assessed using whole-mount staining of the retinal tissue by immunostaining with an anti-β-III tubulin antibody, a commonly used marker of RGCs.

In addition to PTEN conditional knock-out mice we found significant axon regeneration effect in SOCS3 conditional knockouts which was developed by Mori et al (2004, Nature medicine 10, 739-743), among more than 10 mutants tested. In AAV-GFP (control) treated SOCS3f/f mice, a few axonal sprouts could be found close to the crush site, but none of these extended distally beyond 500 μm. AAV-Cre treated SOCS3f/f mice however exhibited significant enhancement in the number of regenerating fibers beyond the crush site. At 14 days post-crush injury, many regenerating axons extended through 500 μm and 1000 μm distally in AAV6 Cre-injected SOCS3f/f mice and some axons were seen up to as far as 1500 um past the crush site.

Analysis of cell viability revealed significant improvement in cell survival following optic nerve crush in SOCS3 deleted mice. In this regard, a drastic cell reduction was found in control (AAV-GFP treated) retinas. AAV-Cre-injected Socs3f/f retinas however showed a clear survival effect as indicated by the number of Tuj1-positive RGCs within stained whole mount retinas.

In addition to AAV-Cre, we performed the optic nerve injury procedure in the SOCS3f/f mice crossed with a Thy1-Cre mouse line (Dewachter et al., 2002 J Neurosci 22, 3445-3453) and observed similar axon regeneration phenotype. However, Cre expression is not limited to neurons and starts during embryonic stages in this line. Thus, our further studies have been focused on the experiments with AAV-Cre to delete SOCS3 in RGCs of the floxed allele. Taken together, our results indicate that SOCS3 deletion in RGCs promote both neuronal survival and axon regeneration following optic crush injury.

To examine the temporal effects of SOCS3 deletion, we assessed axon regeneration in SOCS3 conditional knockout mice at different time points following nerve injury. At 1 day post crush (1 dpc), CTB labeled axons stopped immediately at the crush site, with no labeled fibers found distal to the lesion site. At 3 dpc, a few short sprouts could be seen close to the lesion site. However, at 7 dpc, a more dramatic increase in the number of fibers was observed beyond the crush site. In comparison, the AAV-GFP injected control animals showed no fibers beyond the lesion site at any of these early time points. Glial scar responses were however similar in both control and SOCS3 deleted mice, indicating that SOCS3 deletion in RGCs did not affect glial responses in the lesion site. This result contrasts with the observation of numerous axonal sprouts seen at 3 dpc in PTEN deleted mice (Park et al., 2008, supra), indicating that the majority of axon regeneration in SOCS3 deleted RGCs starts between 3-7 days postinjury.

### Example 2

### CNTF Enhances the Axon Regeneration Promoting Effect of SOCS3 Knockout

We intravitreously injected 1 μl CNTF (1 μg/μl) immediately before injury and 3 days after injury (PBS injection was used as control). PBS injected control animals (SOCS3f/f with AAV-GFP) showed no sign of axon regeneration, while CNTF injection showed limited degree of axon regeneration in these control mice, consistent with previous reports (Muller et al., 2009, Mol Cell Neurosci 41, 233-246). SOCS3 knockout mice, treated with PBS, showed a similar regeneration effect to that observed in noninjected animals. In contrast, CNTF injection resulted in a dramatic increase in both numbers and lengths of regenerating axons in SOCS3 deleted mice. Thus exogenous CNTF further enhances axon regeneration from SOCS deleted neurons following optic nerve injury.

### Example 3

### SOCS3 Pharmacological Inhibition Promotes Optic Nerve Regeneration in Adult Mice

Using the same optic nerve crush injury model we used in Example 1, SOCS3 inhibition by several diverse classes of SOCS3 inhibitors (shRNA, siRNA and several OSC3-SAR-derived high-affinity small molecules) delivered to the target neuron cell bodies provides similar enhanced optic nerve regeneration, confirming the versatility of SOCS3 targeting.

As above regeneration is assessed in the injured optic nerve axons by using cholera toxin beta subunit (CTB), an anterograde tracer, injected into the vitreous of the retina following optic nerve injury two days prior to histological analyses. Neuronal survival is assessed using whole-mount staining of the retinal tissue by immunostaining with an anti-β-III tubulin antibody, a commonly used marker of RGCs.

SOCS3 inhibitors: (a) antisense SOCS3 cDNAs generated using PCR and subcloned into a bicistronic retroviral vectors (e.g. Owaki, et al., J. Immunol. 2006 Mar. 1; 176(5):2773-80); (b) mouse shRNA, HushRNA constructs against mus musculus SOCS3 NM_{—}007707 purchased from OriGene, Rockvillle Md.; (c) dominant negative SOCS3 (F25A); and (d) high-affinity (<uM) small-molecule inhibitors of SOCS3 SAR (Babon et al., J Mol Biol. 2009 Mar. 20; 387(1):162-74; Babon et al., Mol Cell 2006 Apr. 21; 22 (2) 205-16).

### Example 4

### SOCS3 Inhibition by Dominant-Negative SOCS3 Promotes Optic Nerve Regeneration after Traumatic Optic Nerve Injury

Retinal and visual function is assessed in patients with traumatic optic nerve injuries given one subretinal injection of adeno-associated virus (AAV) containing a gene encoding a SOC3 inhibitor protein (AAV2-hSOCS3v2) in the injured eye at low (1·5×10¹¹) vector genomes) for up to 2 years. AAV2-hSOCS3v2 is well tolerated and patients show sustained improvement in subjective and objective measurements of vision (ie, dark adaptometry, pupillometry, electroretinography, nystagmus, and ambulatory behaviour). Patients have at least a 2 log unit increase in pupillary light responses.

Delivery methods were adapted from those of Maguire et al., Lancet. 2009 Nov. 7; 374(9701):1597-605. The AAV2 vector, AAV2-hSOCS3v2 contains a dominant-negative human SOCS3 (F25A) cDNA with a modified Kozak sequence engineered at the translational start site. The cDNA is under control of a hybrid chicken β actin (CBA) promoter. Following generation by transfection of HEK293 cells, the vector is purified by microfluidization, filtration, cation-exchange chromatography, density gradient ultra-centrifugation and final diafiltration into phosphatebuffered saline. This process achieves efficient removal of process- and product-related impurities (empty capsids). The lot of clinical vector prepared is subjected to an extensive series of quality control assays, meeting pre-determined specifications for identity, purity, potency, safety, and stability. The product is supplemented with Pluronic F68 NF Prill Poloxamer 188 (Pluronic F68; BASF, Germany) to prevent subsequent losses of vector to product contact surfaces during storage and administration.

Surgery is performed under general anesthesia using standard vitreoretinal techniques described for subretinal surgery (e.g. Joseph D, Thomas M. Surgical removal of subretinal choroidal neovascular membranes. In: Wilkinson C, ed. Retina (Philadelphia, Pa. Third ed. St. Louis: Mosby, Inc.; 2001:2562-72). After stabilizing the eye with a retrobulbar injection (4.0 mL marcaine 0.25% and 1.0 mL of triamcinolone acetonide (40 mg/ml), a standard 3 port pars plana vitrectomy is performed with removal of the posterior cortical vitreous. The injection cannula is placed in direct apposition to the retina in an area between the fovea and the temporal vascular arcade. We prefer two delivery cannulae: the BD Ophthalmic Systems Visitec™ 32 gauge MVR Cannula (Franklin Lakes, N.J.) and the Bausch & Lomb Storz 39 gauge translocation cannula (San Dimas, Calif.). After lowering the infusion pressure, 1.5×1010 vg AAV2-hSOCS3v2 in a volume of 150 μl is injected into the subretinal space, thereby creating a localized dome-shaped retinal detachment, a “bleb”. A 50% fluid-air exchange is then performed prior to closure of incisions. The subject is recovered from anesthesia but kept in a supine position for 24 hours while the subretinal injection resorbed and the retina reattached. To minimize inflammation resulting from surgery and potential or unexpected immune response to vector, subjects are given 1 mg/kg/day prednisone for a total of 10 days, beginning three days before the vector injection, followed by 0.5 mg/kg/day for an additional 7 days.

Ophthalmic exams include vision testing, slit lamp biomicroscopy, intraocular pressure measurements, and fundoscopy with indirect ophthalmoscopic exam and fundus biomicroscopy. Optical coherence tomography (OCT) is performed with a Zeiss Meditec Stratus optical coherence tomography (OCT3) system (Dublin, Calif.). Fundus photos are taken with a TOPCON Medical systems fundus camera (Paramus, N.J.) and an Optos P200 instrument (Optos Plc, Fife, Scotland). Kinetic visual fields are measured using Goldmann perimetry and electroretinograms (ERGs) are performed. The presence and character of any nystagmus is monitored and recorded on digital videotape.

### Example 5

### Intracerebral Infusion of Diverse SOCS3 Inhibitors Promotes Striatal Neurogenesis after Stroke in Adult Rats

This example demonstrates that intrastriatal infusion of SOCS3 inhibitor in the postischemic period promotes striatal neurogenesis after stroke.

SOCS3 inhibitors: (a) antisense SOCS3 cDNAs generated using PCR are subcloned into a bicistronic retroviral vectors (Owaki, et al., J. Immunol. 2006 Mar. 1; 176(5):2773-80); (b) rat shRNA, HuSH 29mer shRNA constructs against Rat SOCS3 in pRFP-C-RS and pGFP-V-RS vectors (pRFP-C-RS and pGFP-V-RS; OriGene, Rockville, Md.); (c) dominant negative SOCS3 (F25A); and (d) high-affinity (<uM) small-molecule inhibitors of SOCS3 SAR (Babon et al., J Mol Biol. 2009 Mar. 20; 387(1):162-74; Babon et al., Mol Cell 2006 Apr. 21; 22 (2) 205-16)).

Adult rats are subjected to 2-hour middle cerebral artery occlusion (MCAO). Inhibitors are infused into the ischemic striatum either during the first week after MCAO, with the animals being killed directly thereafter, or during the third and fourth weeks, with the rats being killed 1 week later. New cells are labeled with 5′-bromo-2′deoxyuridine (BrdU) on day 7 or during the second week, respectively. Neurogenesis is assessed immunocytochemically with antibodies against BrdU and neuronal, glial, or progenitor markers.

Results show that SOCS3 inhibitor infusion increases cell proliferation in the ipsilateral SVZ and the recruitment of new neuroblasts into the striatum after MCAO and improved survival of new mature neurons.

Our experimental design was adapted from Kokayashi et al., Stroke. 2006; 37:2361-2367. Male Wistar rats (280 to 320 g) were housed under a 12-hour:12-hour light/dark cycle with ad libitum access to food and water. In the first experiment, rats are implanted with infusion cannulas (Alzet brain infusion kit, Durect) connected to subcutaneously placed osmotic minipumps (Alzet model 1007D, Durect), into the right striatum just before induction of the 2-hour MCAO. Coordinates are as follows: 1 mm rostral and 2.5 mm lateral to bregma, 5 mm ventral to dura, and toothbars at −3.3 mm. SOCS3 inhibitors (a)-(c), supra, or phosphate-buffered saline (PBS) are infused (0.5 μL/h) for 7 days. 5′-Bromo-2′deoxyuridine (BrdU, 50 mg/kg; Sigma) is injected intraperitoneally 3 times at 2-hour intervals on day 7, and the animals killed 2 hours thereafter. In the second experiment, BrdU is given 3 times daily for 1 week starting 6 days after the 2-hour MCAO. Inhibitor or vehicle is infused intrastriatally via osmotic minipumps (Alzet, model 2002, Durect) from day 13 to day 26, after which the pumps are removed. The animals are killed 1 week later.

Transient MCAO is induced by the intraluminal filament technique. After being fasted overnight, rats are anesthetized with N20 and 02 (70%:30%) and 1.5% halothane and intubated. A silicone rubber-coated nylon monofilament is inserted into the internal carotid artery. After 2 hours, the filament is withdrawn. During the entire procedure, physiological parameters are maintained within a predetermined range. Body temperature is monitored and regulated for 4 hours after MCAO.

After transcardial perfusion with ice-cold, 4% phosphate-buffered paraformaldehyde (PFA), brains are postfixed in PFA for 24 hours and sectioned coronally at 30 μm on dry ice with use of a microtome. Fluorescence double staining is used for visualization of BrdU and double cortin (Dcx), S10013, or neuronal nuclei (NeuN).

The foregoing examples and detailed description are offered by way of illustration and not by way of limitation. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for treating a mammal in need thereof having a CNS injury, the method comprising the step of: delivering directly to the body of an injured CNS neuron of the mammal an effective amount of a specific inhibitor of suppressor of cytokine signaling 3 (SOCS3).

2. The method of claim 1, wherein the delivery is intracortical, intracerebral, or intra-retinal.

3. The method of claim 1 wherein the delivery is made to cerebrospinal fluid at the body of the injured CNS neuron.

4. The method of claim 1 wherein the delivery is effected with a cannula.

5. The method of claim 1, wherein the inhibitor is SOCS3-specific hpRNA or siRNA.

6. The method of claim 1 wherein the inhibitor is antisense SOCS3 or dominant negative SOCS3.

7. The method of claim 1 wherein the neuron is injured as a result of trauma or stroke.

8. The method of claim 1 further comprising delivering to the body of the injured neuron cytokine ciliary neurotrophic factor (CNTF) sufficient to activate gp 130 in the neuron.

9. The method of claim 1 further comprising detecting a resultant improved recovery from the injury.

10. The method of claim 1, wherein the delivery is intra-retinal, the inhibitor is SOCS3-specific hpRNA or siRNA, the delivery is effected with a cannula, and the method further comprising detecting a resultant improved recovery from the injury.

11. A method for promoting regeneration of an injured CNS neuron in a mammal in need thereof, the method comprising the step of: delivering directly to the body of the injured CNS neuron an effective amount of a specific inhibitor of suppressor of cytokine signaling 3 (SOCS3), wherein the delivered SOCS3 inhibitor promotes regeneration of the injured CNS neuron.

12. The method of claim 11, further comprising the step of detecting a resultant promoted regeneration of the injured CNS neuron.

13. An intracerebral or intraretinal cannula loaded with an amount of a specific inhibitor of SOCS3 effective to promote regeneration of an injured CNS neuron when delivered directly to the body of the injured CNS neuron in a mammal in need thereof.

14. The method of claim 1, wherein the inhibitor is SOCS3-specific hpRNA.

15. The method of claim 1 wherein the mammal is mouse, and delivery is intravitreal following optic nerve injury, the method further comprising detecting a resultant improved recovery from the injury.

16. The method of claim 15, wherein the inhibitor is SOCS3-specific hpRNA.

17. The method of claim 15, wherein the inhibitor is SOCS3-specific hpRNA that is HuSH 29mer shRNA constructs against mouse SOCS3 in pRFP-C-RS and pGFP-V-RS vectors.

18. The method of claim 1 wherein the mammal is rat, and delivery is by infusion into ischemic striatum after middle cerebral artery occlusion (MCAO), the method further comprising detecting a resultant improved recovery from the injury.

19. The method of claim 18, wherein the inhibitor is SOCS3-specific hpRNA.

20. The method of claim 18, wherein the inhibitor is SOCS3-specific hpRNA that is HuSH 29mer shRNA constructs against rat SOCS3 in pRFP-C-RS and pGFP-V-RS vectors.
